(19) United States
(12) Patent Application Publication
(10) Pub. No.: US 2025/0135493 A1
(43) Pub. Date: May 1, 2025

(54) SYSTEMS AND METHODS FOR A TRANSDUCER ASSEMBLY

(71) Applicant: GE Precision Healthcare LLC, Waukesha, WI (US)

(72) Inventors: Manon Chateauvieux, Valbonne (FR); Alexis Hubert, Valbonne (FR); Sihem Lazizi, Valbonne (FR); Solenne Drewniak, Valbonne (FR); Fereshteh Ordokhani, Valbonne (FR)

(21) Appl. No.: 18/500,011

(22) Filed: Nov. 1, 2023

(51) Int. Cl. B06B 1/06; A61B 8/00; H10N 30/072; H10N 30/088; H10N 30/87

(52) U.S. Cl. CPC B06B 1/067; H10N 30/072; H10N 30/088; H10N 30/875; A61B 8/4488; B06B 2201/76

(57) ABSTRACT

Systems and methods are provided for a transducer assembly. In one example the transducer assembly includes a flexible circuit and a transducer, where the transducer incudes a piezoelectric layer and an impedance matching layer. The transducer includes a plurality of transducer elements formed via a plurality of dicing kerfs positioned between neighboring transducer elements of the plurality of transducer elements, where each dicing kerf of the plurality of dicing kerfs extends only partially through the impedance matching layer.

### FIELD

Embodiments of the subject matter disclosed herein relate to transducer assemblies, and more specifically to methods of manufacturing transducer assemblies.

### BACKGROUND

Certain ultrasonic transducer assemblies are typically employed in applications including non-destructive evaluation (NDE) and medical diagnostic imaging, such as ultrasound applications. The ultrasonic transducer assembly generally includes an array of ultrasonic transducer elements coupled to an electronics array (e.g., a flexible circuit).

Generally, the ultrasonic transducer assembly includes a transducer with hundreds or thousands of individual transducer elements. Piezoelectric transducers (e.g., lead zirconate titante transducers) are a widely used type of ultrasonic transducer. Piezoelectric transducers generally include a piezoelectric material capable of changing physical dimensions when subjected to electrical or mechanical stress. In addition, piezoelectric transducers may include layers of impedance matching materials and layers of impedance dematching materials. A flexible circuit may be electrically coupled to the transducer elements to provide electrical control of the transducer for beam forming, signal amplification, control functions, signal processing, etc.

Large arrays of transducer elements may be formed by dicing a transducer into rows and/or columns. The array of transducer elements may be one-dimensional (1D) (e.g., a linear array or row of transducer elements) for two-dimensional (2D) imaging. Similarly, the array may be 2D for three-dimensional (3D) imaging (e.g., volumetric imaging). Each transducer assembly comprises a subarray of transducer elements and a flexible circuit coupled to the transducer elements.

### BRIEF DESCRIPTION

In one embodiment, a transducer assembly includes a flexible circuit and a transducer, where the transducer incudes a piezoelectric layer and an impedance matching layer. The transducer includes a plurality of transducer elements formed via a plurality of dicing kerfs positioned between neighboring transducer elements of the plurality of transducer elements, where each dicing kerf of the plurality of dicing kerfs extends only partially through the impedance matching layer.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present disclosure will be better understood from reading the following description of non-limiting embodiments, with reference to the attached drawings, wherein below:

FIG. 1 shows a schematic diagram of an ultrasound imaging system;

FIG. 2 shows a composite wafer including dicing kerfs oriented in two orthogonal directions;

FIG. 3 shows the composite wafer of FIG. 2 divided into transducers;

FIG. 4 shows a transducer of FIG. 3 and a flexible circuit;

FIG. 5 shows a cross-sectional view of an example transducer before dicing;

FIG. 6 shows a first cross-sectional view of an example transducer after dicing;

FIG. 7 shows a cross-sectional view of an example transducer assembly including the diced transducer of FIG. 6 and a flexible circuit including ground recovery areas;

FIG. 8 shows a two-dimensional transducer including ground recovery areas;

FIG. 9 shows a method for manufacturing a one-dimensional transducer probe;

FIG. 10 shows a method for manufacturing a two-dimensional transducer probe;

FIG. 11 shows a composite wafer including a piezoelectric layer;

FIG. 12 shows a composite wafer including dicing kerfs oriented in a single direction;

FIG. 13 shows a second cross-sectional view of an example transducer after dicing;

FIG. 14 shows a first one-dimensional transducer including ground recovery areas;

FIG. 15 shows a second one-dimensional transducer including ground recovery areas; and

FIG. 16 shows a cross-sectional view of another example transducer assembly including a diced transducer and a flexible circuit including ground recovery areas.

### DETAILED DESCRIPTION

This description and embodiments of the subject matter disclosed herein relate to a transducer assembly and methods and systems for manufacturing the transducer assembly. Typically, in the manufacturing of ultrasound (e.g., transducer) probes, transducers are coupled to flexible circuits before being diced into transducer elements. As such, each transducer is diced individually as a part of a transducer assembly. In this way, dicing equipment occupation may be increased as compared to a method that dices multiple transducers collectively. Further, an electrically conductive material is often added to the transducer assembly as a continuous film or layer after dicing has occurred to ensure ground recovery of the transducer assembly. A deposition of an additional layer or film increases both the manufacturing complexity and material cost of each ultrasound probe.

Thus, embodiments are provided herein for manufacturing a transducer assembly of an ultrasound probe that allows for multiple transducers to be diced collectively, where each transducer assembly includes an electrically conductive material for ground recovery. A composite wafer may be diced and then separated into individual transducers, with each transducer configured for an ultrasound probe. By dicing the transducers collectively as a part of a composite wafer, dicing time may be reduced by decreasing the number of times transducers are loaded and/or unloaded from dicing equipment. Further, at least a portion of an impedance matching layer of each transducer may not be diced, resulting in a continuous layer that extends entirely across the transducer. As such, the impedance matching layer of the transducer may provide mechanical support and may include an electrically conductive material for ground recovery of the transducer. In this way, an additional conductive layer/film may not be deposited onto each transducer, reducing manufacturing costs and complexity.

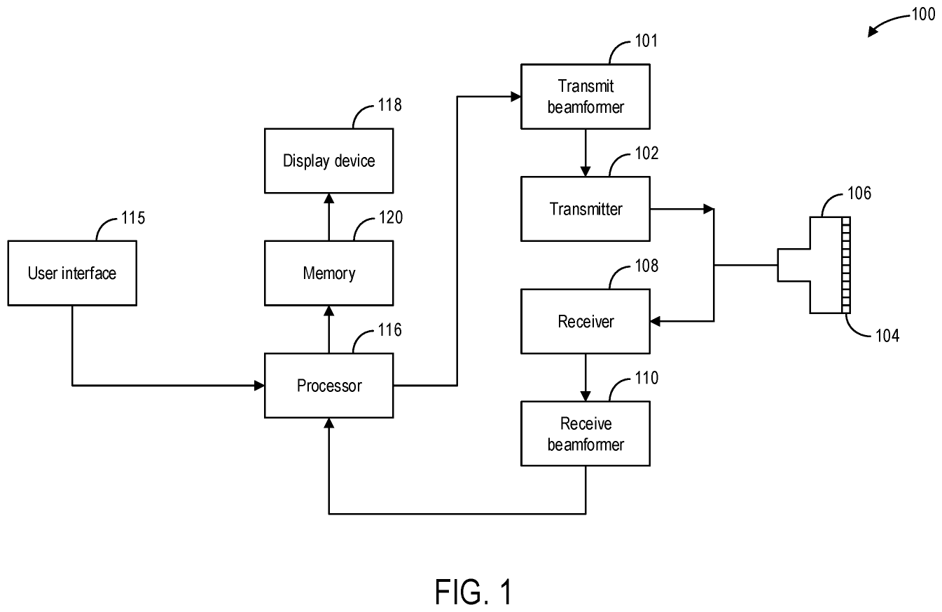

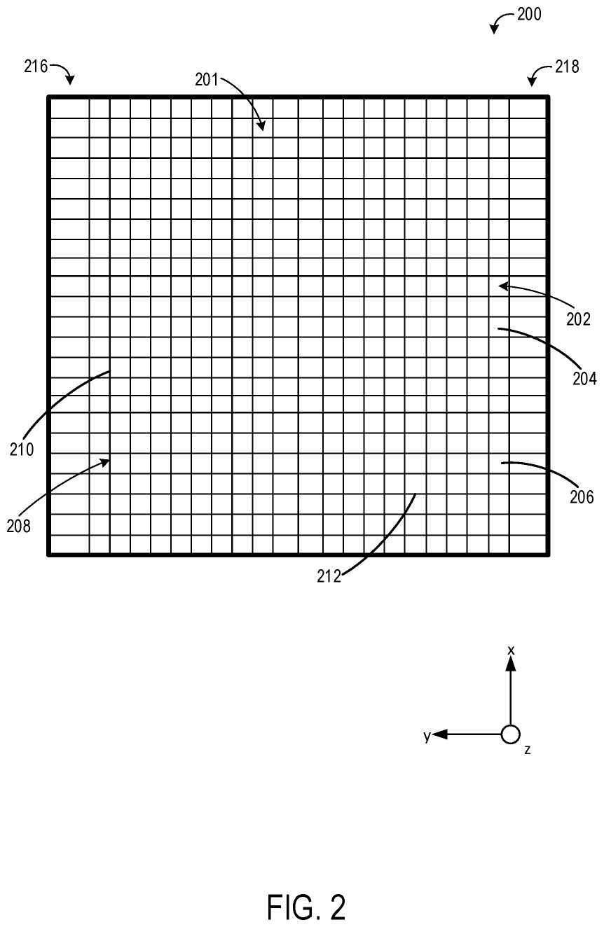

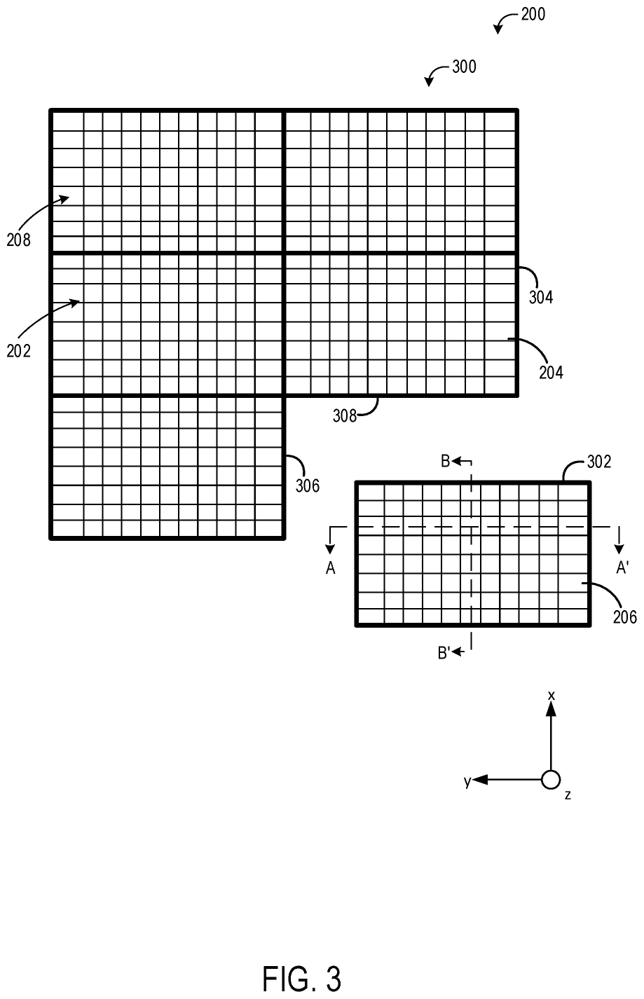

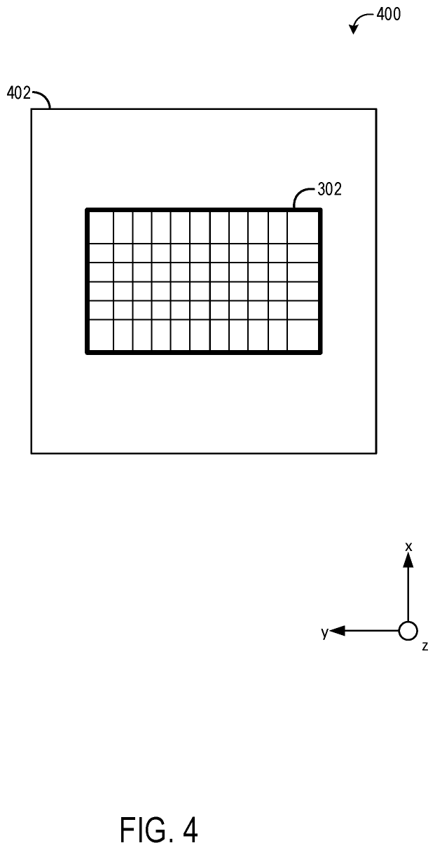

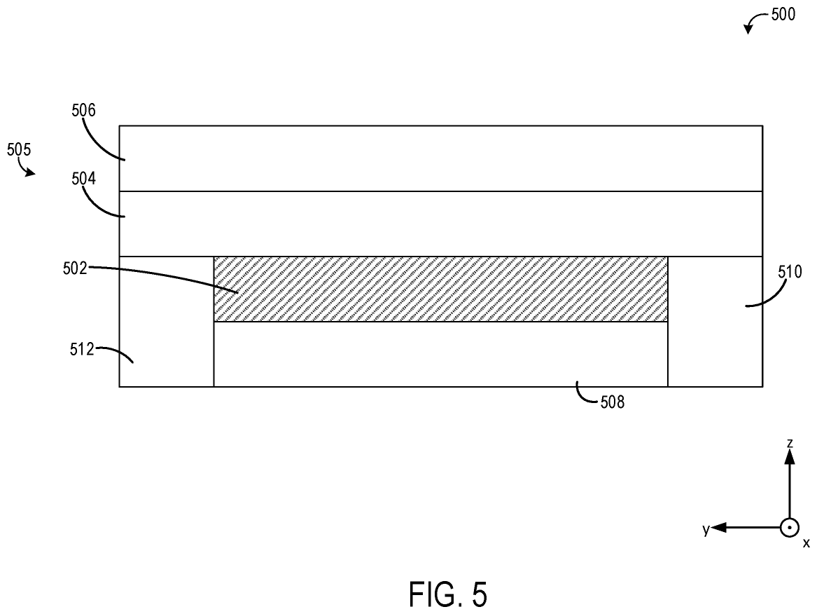

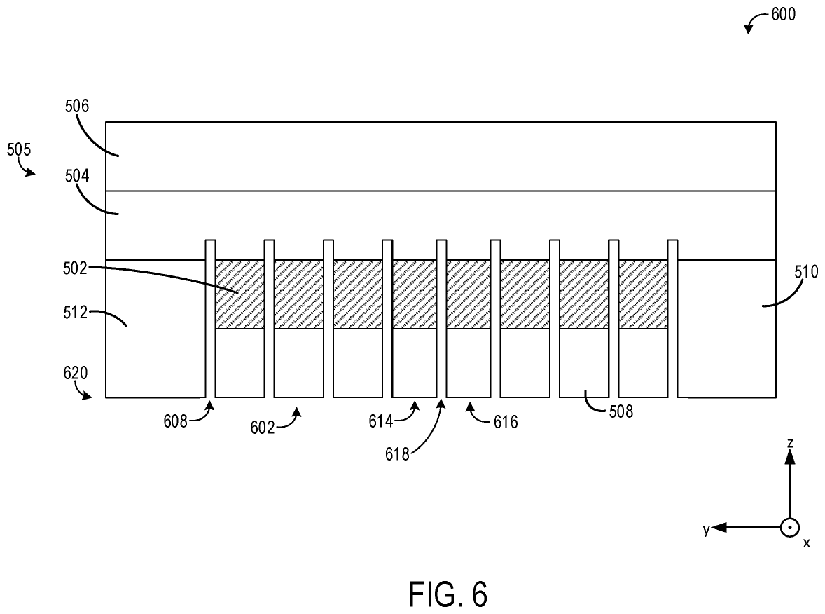

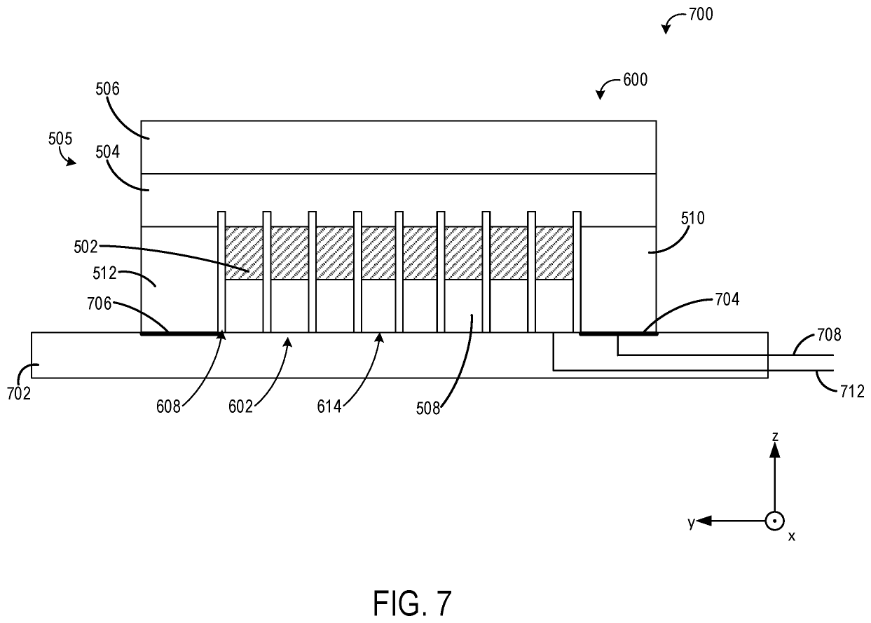

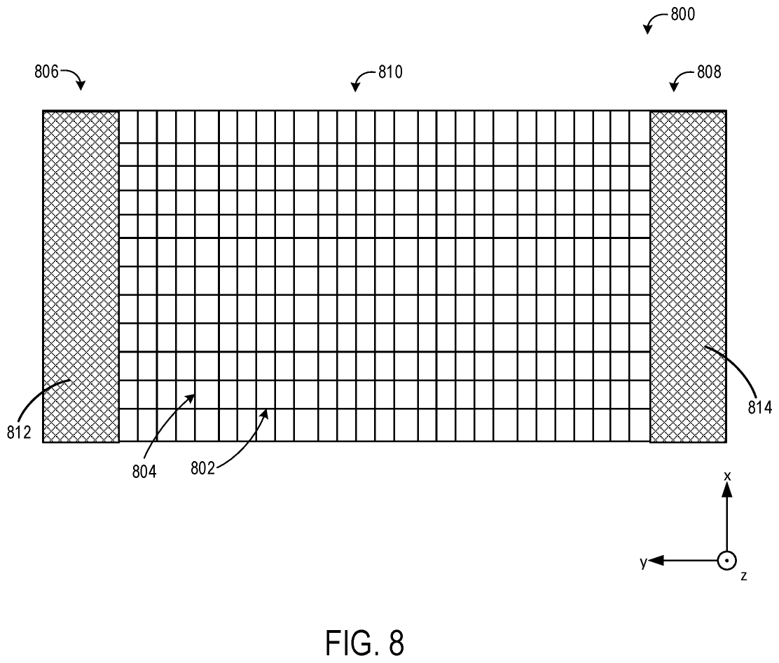

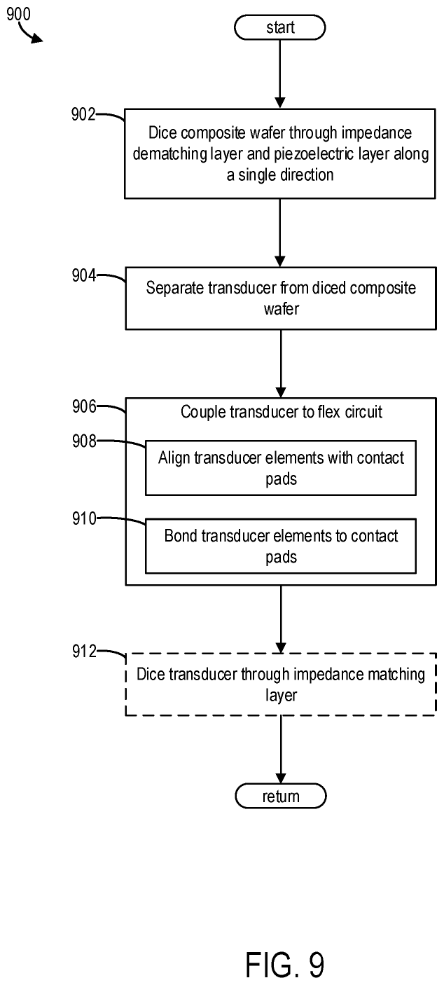

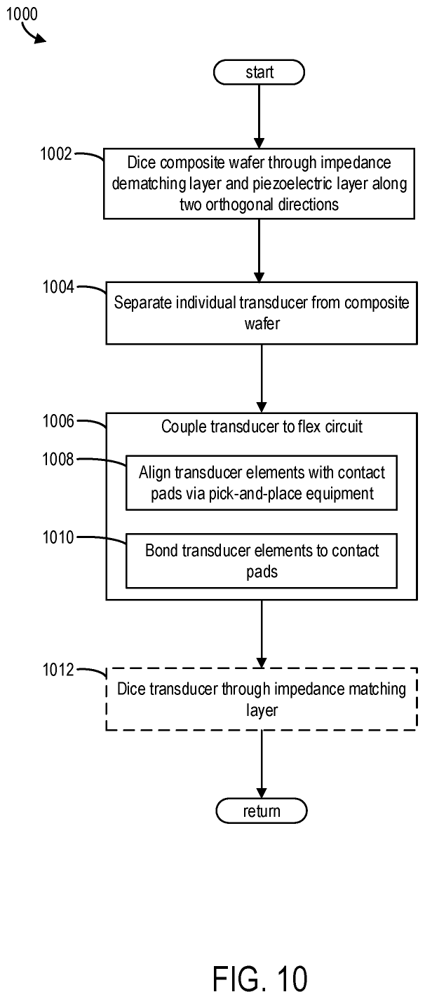

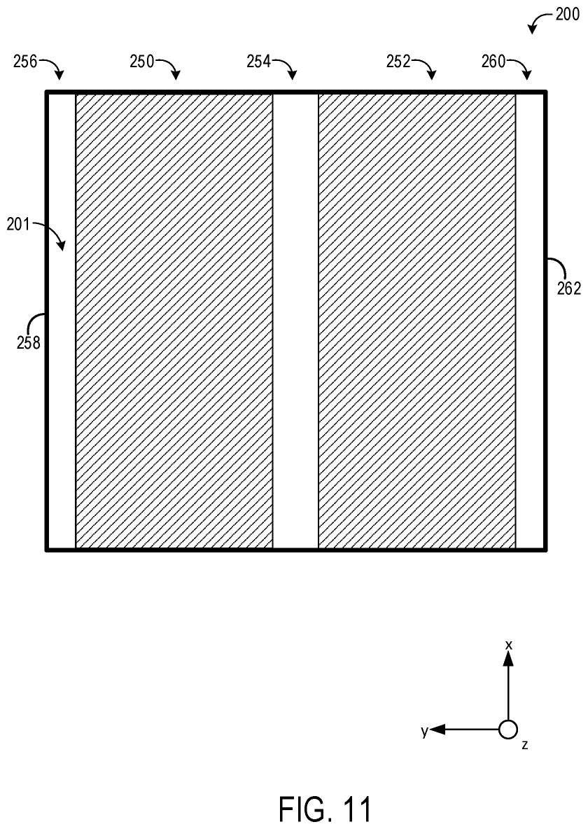

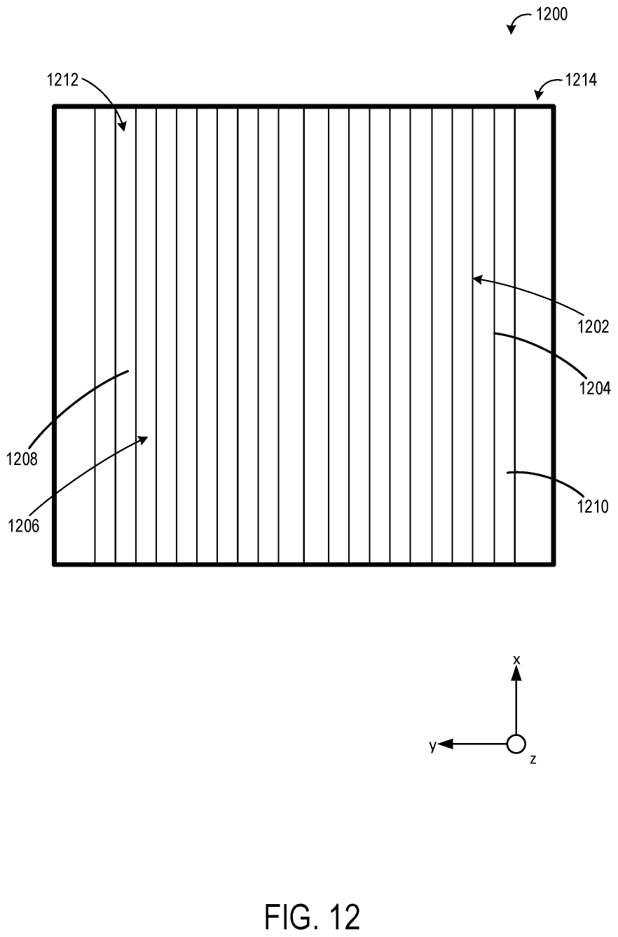

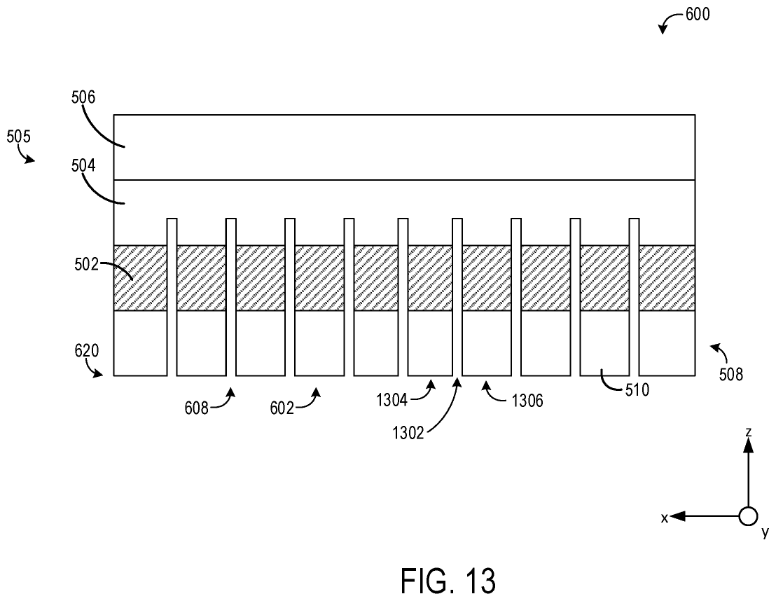

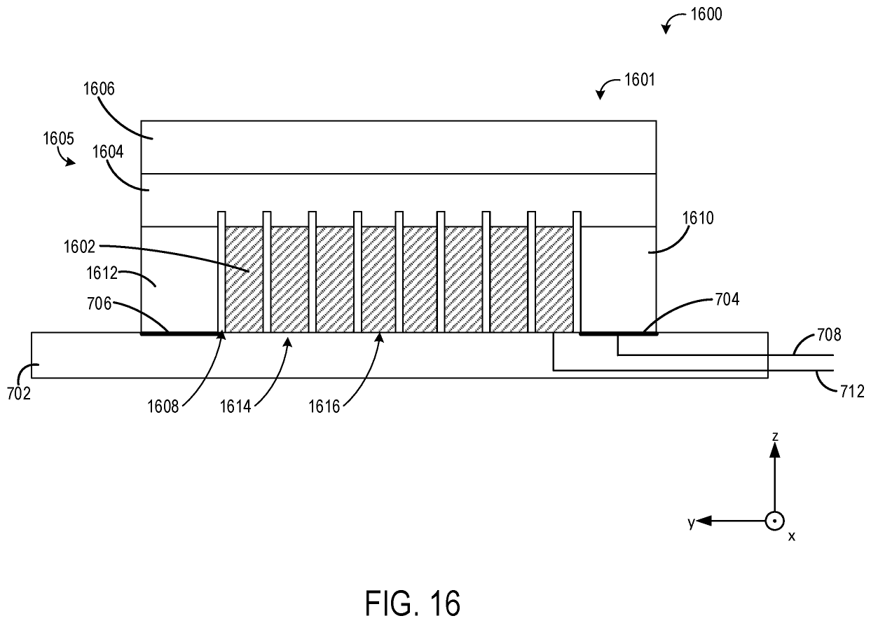

The transducer assembly may be utilized in diagnostic imaging, and may be integrated into an imaging system, such as the ultrasound imaging system shown in FIG. 1. The transducer assembly may include a transducer taken from a composite wafer and bonded to a flexible circuit (e.g., an electronics array). The composite wafer may include an impedance matching layer, as well as an impedance dematching layer and a piezoelectric layer, as illustrated in FIG. 11. Diced composite wafers including hundreds or thousands, in some examples, of transducer elements are shown in FIGS. 2 and 12. FIG. 2 illustrates a composite wafer that has been diced in two orthogonal directions while FIG. 12 illustrates a composite wafer that has been diced in a single direction. The composite wafers may include one or more transducers, with each transducer including an array of the diced transducer elements. Each transducer may be removed from a composite wafer, as shown in FIG. 3, and bonded to a flexible circuit, as shown in FIG. 4. FIG. 5 illustrates a transducer before dicing, which may include the impedance matching layer, the piezoelectric layer, and the impedance dematching layer. Dicing kerfs may extend into the transducer, extending entirely through the piezoelectric layer and the impedance dematching layer, and extending only partially through the impedance matching layer, as shown in FIGS. 6 and 13. The dicing kerfs may create individual transducer elements, with each transducer element separated from neighboring transducer elements by one or more of the dicing kerfs. Thus, in some examples, each transducer element may include the impedance dematching layer positioned under the piezoelectric layer. However, in other examples, as illustrated in FIG. 16, the impedance dematching layer under the piezoelectric layer may be omitted. Each of the transducer elements may be aligned with and bonded to a contact pad of a flexible circuit (e.g., a printed circuit board), as illustrated in FIG. 7. Alignment of the transducer elements with the contact pads may be facilitated by pick-and-place equipment to ensure proper electrical connections. The transducer elements may be arranged within a center section of the transducer, and two ends of the transducer may include ground recovery areas, as shown in FIGS. 8, 14, and 15. The ground recovery areas of the transducer may align with ground signal connections of the flexible circuit such that the transducer assembly is grounded. Further, the ground recovery areas may be oriented such that a desired imaging footprint is achieved. A method for manufacturing a one-dimensional transducer assembly and a method for manufacturing a two-dimensional transducer assembly are illustrated in FIGS. 9 and 10, respectively.

FIG. 1 shows a schematic diagram of an ultrasound imaging system 100 in accordance with an embodiment of the disclosure. The ultrasound imaging system 100 includes a transmit beamformer 101 and a transmitter 102 that drives elements (e.g., transducer elements) 104 within a transducer assembly, herein referred to as probe 106, to emit pulsed ultrasonic signals (referred to herein as transmit pulses) into a body (not shown). According to an embodiment, the probe 106 may be a one-dimensional transducer assembly probe. However, in some embodiments, the probe 106 may be a two-dimensional matrix transducer assembly probe. As explained further below, the transducer elements 104 may be comprised of a piezoelectric material. When a voltage is applied to a piezoelectric crystal, the crystal physically expands and contracts, emitting an ultrasonic spherical wave. In this way, transducer elements 104 may convert electronic transmit signals into acoustic transmit beams.

After the transducer elements 104 of the probe 106 emit pulsed ultrasonic signals into a body (of a patient), the pulsed ultrasonic signals are back-scattered from structures within an interior of the body, like blood cells or muscular tissue, to produce echoes that return to the transducer elements 104. The echoes are converted into electrical signals, or ultrasound data, by the transducer elements 104 and the electrical signals are received by a receiver 108. The electrical signals representing the received echoes are passed through a receive beamformer 110 that outputs ultrasound data. Additionally, transducer elements 104 may produce one or more ultrasonic pulses to form one or more transmit beams in accordance with the received echoes.

According to some embodiments, the probe 106 may contain electronic circuitry to do all or part of the transmit beamforming and/or the receive beamforming. For example, all or part of the transmit beamformer 101, the transmitter 102, the receiver 108, and the receive beamformer 110 may be situated within the probe 106. The terms “scan” or “scanning” may also be used in this disclosure to refer to acquiring data through the process of transmitting and receiving ultrasonic signals. The term “data” may be used in this disclosure to refer to either one or more datasets acquired with an ultrasound imaging system. In one embodiment, data acquired via ultrasound imaging system 100 may be used to train a machine learning model. A user interface 115 may be used to control operation of the ultrasound imaging system 100, including to control the input of patient data (e.g., patient medical history), to change a scanning or display parameter, to initiate a probe repolarization sequence, and the like. The user interface 115 may include one or more of the following: a rotary element, a mouse, a keyboard, a trackball, hard keys linked to specific actions, soft keys that may be configured to control different functions, and a graphical user interface displayed on a display device 118.

The ultrasound imaging system 100 also includes a processor 116 to control the transmit beamformer 101, the transmitter 102, the receiver 108, and the receive beamformer 110. The processor 116 is in electronic communication (e.g., communicatively connected) with the probe 106. For purposes of this disclosure, the term “electronic communication” may be defined to include both wired and wireless communications. The processor 116 may control the probe 106 to acquire data according to instructions stored on a memory of the processor, and/or memory 120. The processor 116 controls which of the transducer elements 104 are active and the shape of a beam emitted from the probe 106. The processor 116 is also in electronic communication with the display device 118, and the processor 116 may process the data (e.g., ultrasound data) into images for display on the display device 118. The processor 116 may include a central processor (CPU), according to an embodiment. According to other embodiments, the processor 116 may include other electronic components capable of carrying out processing functions, such as a digital signal processor, a field-programmable gate array (FPGA), or a graphic board. According to other embodiments, the processor 116 may include multiple electronic components capable of carrying out processing functions. For example, the processor 116 may include two or more electronic components selected from a list of electronic components including: a central processor, a digital signal processor, a field-programmable gate array, and a graphic board. According to another embodiment, the processor 116 may also include a complex demodulator (not shown) that demodulates the RF data and generates raw data. In another embodiment, the demodulation can be carried out earlier in the processing chain. The processor 116 is adapted to perform one or more processing operations according to a plurality of selectable ultrasound modalities on the data. In one example, the data may be processed in real-time during a scanning session as the echo signals are received by receiver 108 and transmitted to processor 116. For the purposes of this disclosure, the term “real-time” is defined to include a procedure that is performed without any intentional delay. For example, an embodiment may acquire images at a real-time rate of 7-20 frames/sec. The ultrasound imaging system 100 may acquire 2D data of one or more planes at a significantly faster rate. However, it should be understood that the real-time frame-rate may be dependent on the length of time that it takes to acquire each frame of data for display. Accordingly, when acquiring a relatively large amount of data, the real-time frame-rate may be slower. Thus, some embodiments may have real-time frame-rates that are considerably faster than 20 frames/see while other embodiments may have real-time frame-rates slower than 7 frames/sec. The data may be stored temporarily in a buffer (not shown) during a scanning session and processed in less than real-time in a live or off-line operation. Some embodiments of the invention may include multiple processors (not shown) to handle the processing tasks that are handled by processor 116 according to the exemplary embodiment described hereinabove. For example, a first processor may be utilized to demodulate and decimate the RF signal while a second processor may be used to further process the data, for example by augmenting the data as described further herein, prior to displaying an image. It should be appreciated that other embodiments may use a different arrangement of processors.

The ultrasound imaging system 100 may continuously acquire data at a frame-rate of, for example, 10 Hz to 30 Hz (e.g., 10 to 30 frames per second). Images generated from the data may be refreshed at a similar frame-rate on display device 118. Other embodiments may acquire and display data at different rates. For example, some embodiments may acquire data at a frame-rate of less than 10 Hz or greater than 30 Hz depending on the size of the frame and the intended application. A memory 120 is included for storing processed frames of acquired data. In an exemplary embodiment, the memory 120 is of sufficient capacity to store at least several seconds' worth of frames of ultrasound data. The frames of data are stored in a manner to facilitate retrieval thereof according to its order or time of acquisition. The memory 120 may comprise any known data storage medium.

In various embodiments of the present invention, data may be processed in different mode-related modules by the processor 116 (e.g., B-mode, Color Doppler, M-mode, Color M-mode, spectral Doppler, Elastography, TVI, strain, strain rate, and the like) to form 2D or 3D data. For example, one or more modules may generate B-mode, color Doppler, M-mode, color M-mode, spectral Doppler, Elastography, TVI, strain, strain rate, and combinations thereof, and the like. As one example, the one or more modules may process color Doppler data, which may include traditional color flow Doppler, power Doppler, HD flow, and the like. The image lines and/or frames are stored in memory and may include timing information indicating a time at which the image lines and/or frames were stored in memory. The modules may include, for example, a scan conversion module to perform scan conversion operations to convert the acquired images from beam space coordinates to display space coordinates. A video processor module may be provided that reads the acquired images from a memory and displays an image in real time while a procedure (e.g., ultrasound imaging) is being performed on a patient. The video processor module may include a separate image memory, and the ultrasound images may be written to the image memory in order to be read and displayed by display device 118.

In various embodiments of the present disclosure, one or more components of ultrasound imaging system 100 may be included in a portable, handheld ultrasound imaging device. For example, display device 118 and user interface 115 may be integrated into an exterior surface of the handheld ultrasound imaging device, which may further contain processor 116 and memory 120. Probe 106 may comprise a handheld probe in electronic communication with the handheld ultrasound imaging device to collect raw ultrasound data. Transmit beamformer 101, transmitter 102, receiver 108, and receive beamformer 110 may be included in the same or different portions of the ultrasound imaging system 100. For example, transmit beamformer 101, transmitter 102, receiver 108, and receive beamformer 110 may be included in the handheld ultrasound imaging device, the probe, and combinations thereof.

After performing a two-dimensional ultrasound scan, a block of data comprising scan lines and their samples is generated. After back-end filters are applied, a process known as scan conversion is performed to transform the two-dimensional data block into a displayable bitmap image with additional scan information such as depths, angles of each scan line, and so on. During scan conversion, an interpolation technique is applied to fill missing holes (e.g., pixels) in the resulting image. These missing pixels occur because each element of the two-dimensional block typically covers many pixels in the resulting image. For example, in current ultrasound imaging systems, a bicubic interpolation is applied which leverages neighboring elements of the two-dimensional block. As a result, if the two-dimensional block is relatively small in comparison to the size of the bitmap image, the scan-converted image will include areas of poor or low resolution, especially for areas of greater depth.

FIG. 2 shows a composite wafer 200 that has been diced. FIGS. 2-8 and 11-16 include a coordinate system to orient the views. The z-axis may be a vertical axis (e.g., parallel to a gravitational axis), the y-axis may be a longitudinal axis (e.g., horizontal axis), and/or the x-axis may be a lateral axis, in one example. However, the axes may have other orientations, in other examples.

The composite wafer 200 may include a piezoelectric layer, an impedance matching layer, and an impedance dematching layer 201 (in the view shown in FIG. 2, the piezoelectric layer and the impedance matching layer are positioned under the impedance dematching layer and hence are not visible in FIG. 2). The piezoelectric layer may be positioned intermediate at least a portion of the impedance matching layer and at least a portion of the impedance dematching layer 201, relative to the z-axis.

Turning to FIG. 11, a positioning of the piezoelectric layer of the composite wafer 200 is illustrated. The positioning of the piezoelectric layer within the composite wafer 200 is represented by a first piezoelectric section 250 and a second piezoelectric section 252. The first piezoelectric section 250 and the second piezoelectric section 252 may each extend across a length of the composite wafer 200, relative to the x-axis. A portion 254 of conductive ground material may be positioned between the first piezoelectric section 250 and the second piezoelectric section 252, relative to the y-axis. Further, a portion 256 of conductive ground material may be positioned between the first piezoelectric section 250 and a first edge 258 of the composite wafer 200. Similarly, a portion 260 of conductive ground material may be positioned between the second piezoelectric section 252 and a second edge 262 of the composite wafer 200, where the second edge 262 is opposite the first edge 258, relative to the y-axis.

The composite wafer 200 is configured such the composite wafer may be divided into one or more transducers that may each be included in an ultrasound probe (e.g., probe 106 of FIG. 1). For example, as illustrated in FIG. 2, the composite wafer 200 may be cut (e.g., via a saw, a laser, etc.) into multiple transducers. Dicing of the composite wafer 200 may be performed using a standard dicing saw or via laser dicing, in some examples. In other examples, a deep reactive ion or other semiconductor type etch may be used with an intervening masking step. As a result of dicing, the composite wafer 200 may include a plurality of dicing kerfs 208. The composite wafer 200 may be diced such that the dicing kerfs 208 extend in two orthogonal directions. In this way, the composite wafer 200 may be separated into transducers for one-dimensional ultrasound probes configured for two-dimensional imaging.

The dicing kerfs 208 may include dicing kerfs parallel to the x-axis, such as a second dicing kerf 210, and dicing kerfs parallel to the y-axis, such as a third dicing kerf 212. As such, the second dicing kerf 210 and the third dicing kerf 212 may be oriented orthogonally to one another. The dicing kerfs 208 divide a portion of the composite wafer 200 into a plurality of transducer elements 202, such as a first transducer element 204 and a second transducer element 206. Each of the transducer elements 202 may include a portion of the impedance dematching layer 201 stacked on top of a portion of the piezoelectric layer, relative to the z-axis. The dicing kerfs 208 may separate the transducer elements 202 from neighboring transducer elements. In the illustrated example, each of the transducer elements 202 may have a square or rectangular cross-section parallel to the x-y plane. The composite wafer 200 may be diced into hundreds or thousands of transducer elements, in some examples.

The dicing kerfs 208 may further separate the transducer elements 202 from ground recovery areas of the composite wafer, such as a first ground recovery area 216 and a second ground recovery area 218. The ground recovery areas may correspond to portions of the composite wafer 200 without a piezoelectric layer, such as the portions 254, 256, and/or 260 of FIG. 11. For example, as illustrated in FIG. 2, the first ground recovery area 216 and the second ground recovery area 218 may be positioned along one edge of the composite wafer 200 and in the center of composite wafer relative to the y-axis, respectively, and may each extend entirely across the composite wafer, parallel to the x-axis. Each of the ground recovery areas may include an electrically conductive material to facilitate grounding of the transducer elements 202.

The composite wafer 200 may be diced starting from the impedance dematching layer 201 of the composite wafer. As such, the dicing kerfs 208 may extend entirely through the impedance dematching layer 201, relative to the z-axis. Further, the dicing kerfs 208 may extend entirely through the piezoelectric layer, relative to the z-axis. As such, the dicing kerfs 208 may extend deep enough to separate neighboring transducer elements from one another, where each transducer element includes a portion of the piezoelectric layer stacked on top of a portion of the impedance dematching layer 201, relative to the z-axis. However, the dicing kerfs 208 may only extend partially through the impedance matching layer, in some examples, and may not extend into the impedance matching layer at all, in other examples. As such, the dicing kerfs 208 do not extend through an entirety of the impedance matching layer, and may terminate either within the impedance matching layer or before reaching the impedance matching layer. In this way, the impedance matching layer may provide a common mechanical support for the transducer elements 202. Further, the impedance matching layer may include an electrically conductive material for ground recovery of the transducer elements 202. As such, a conductive film and/or a conductive layer may not be deposited onto the composite wafer 200 after dicing, reducing the number of manufacturing steps (e.g., manufacturing complexity).

FIG. 3 schematically shows the composite wafer 200 including a plurality of transducers 300. In the illustrated example the plurality of transducers 300 (e.g., the composite wafer 200) includes six transducers, such as a first transducer 302 and a second transducer 304. In other examples, the plurality of transducers 300 may include more or less than six transducers. Each transducer includes a portion of the transducer elements 202, with neighboring transducer elements being separated by the dicing kerfs 208. Further, each transducer includes a portion of one or more of the ground recovery areas, such as a portion of the second ground recovery area 218 of FIG. 2.

As shown in FIG. 3, the first transducer 302 may be removed from the rest of the composite wafer 200, such as the second transducer 304. One or more deep dicing kerfs of the composite wafer 200 may extend entirely through the composite wafer, relative to the z-axis, such that the deep dicing kerfs may separate the composite wafer into the transducers 300. For example, a first deep dicing kerf 306 and a second deep dicing kerf 308 may extend entirely through the composite wafer 200, relative to the z-axis, and may separate the first transducer 302 from the rest of the composite wafer. The separation between the first transducer 302 and the rest of the composite wafer, such as the second transducer 304, created by the first deep dicing kerf 306 and the second deep dicing kerf 308, allows the first transducer be removed from the composite wafer. Once removed, the first transducer 302 may be coupled to a flexible circuit, as illustrated in FIG. 4.

FIG. 4 shows a transducer assembly 400 including the first transducer 302 of FIG. 3 coupled to a flexible circuit 402. The flexible circuit 402 may be an electronics array configured to receive electrical signals from the first transducer 302. In some examples, the flexible circuit 402 may be a printed circuit board (PCB). The first transducer 302 may be aligned with the flexible circuit 402 by pick-and-place equipment. As such, individual transducer elements of the first transducer 302 may be properly aligned with contact pads of the flexible circuit 402. In some examples, the first transducer 302 may be diced again after being attached to the flexible circuit 402 to enhance directivity and reduce acoustic crosstalk.

FIG. 5 shows a cross-sectional view of an example transducer 500. The transducer 500 includes a piezoelectric layer 502. In some examples, the piezoelectric layer 502 includes single crystal lead zirconate titante (PZT).

The transducer 500 further includes an impedance matching layer 505. The impedance matching layer 505 may have an acoustic impedance between that of water and that of the piezoelectric layer 502. As such, the impedance matching layer 505 may provide an acoustic impedance gradient for sound waves to smoothly penetrate body tissue and return to the piezoelectric layer 502 for detection. Additionally, in some examples, the impedance matching layer 505 may include ceramics, silicon, flexible organic polymers, metal filled graphite, ceramic powder filled epoxy, glass, and glass-ceramics. Further, the impedance matching layer 505 may include a first sublayer 504 and a second sublayer 506. The second sublayer 506 may be positioned on top of the first sublayer 504, and at least a portion of the first sublayer may be positioned on top of the piezoelectric layer 502, relative to the z-axis. In some examples, the second sublayer 506 may be omitted from the transducer 500, and as such the impedance matching layer 505 may include a single layer (e.g., the first sublayer 504). The first sublayer 504 and/or the second sublayer 506 may include electrically conductive material for ground recovery of the transducer 500.

The transducer 500 further includes an impedance dematching layer 508, a first end section 510, and a second end section 512. The dematching layer 508 is positioned intermediate the two end sections, relative to the y-axis. As such, the piezoelectric layer 502 may be positioned intermediate the first end section 510 and the second end section 512, relative to the y-axis. Further, the piezoelectric layer 502 may be positioned above a portion of the impedance dematching layer 508, relative to the z-axis.

In some examples, the impedance dematching layer 508 may include dense, high modulus metals, such as molybdenum or tungsten, and/or high density ceramics, such as tungsten carbide. The impedance dematching layer 508 may have an acoustic impedance greater than that of the piezoelectric layer 502. As such, the impedance dematching layer 508 may reduce and/or prevent backward emitted sound waves from echoing (e.g., ringing back) into the piezoelectric layer 502 for detection. The first end section 510 and the second end section 512 may be comprised of a conductive material that, at least in some examples, is a different material than the impedance dematching layer. Further, while FIG. 5 shows the first end section 510 and the second end section 512 extending from the impedance matching layer to the bottom of the transducer, in some examples, the impedance dematching layer 508 may extend across an entirety of the bottom of the transducer and the first end section 510 and the second end section 512 may extend from the impedance matching layer to the impedance dematching layer. Because the first end section and the second end section may facilitate ground recovery, the first end section and the second end section may also be referred to herein as a first ground recovery area and a second ground recovery area.

FIG. 6 shows a cross-sectional view of an example transducer 600 after dicing. The cross-sectional view shown in FIG. 6 is taken along the cutting plane A-A′ of FIG. 3, and the transducer 600 may be a non-limiting example of the first transducer 302 of FIG. 3. The transducer 600 includes the piezoelectric layer 502, the impedance matching layer 505, the impedance dematching layer 508, the first end section 510 (e.g., ground conductive material), and the second end section 512 of the transducer 500 of FIG. 5 as well as a plurality of dicing kerfs 608. The piezoelectric layer 502 may be positioned between the first end section 510 and the second end section 512, relative to the y-axis. As such, the piezoelectric layer 502 may not extend entirely across the transducer 600, relative to the y-axis.

The plurality of dicing kerfs 608 may extend into the transducer 600 from a bottom side 620 of the transducer. The plurality of dicing kerfs 608 may be a non-limiting example of the dicing kerfs 208 of FIG. 2. The dicing kerfs 608 may extend all the way through the impedance dematching layer 508 and the piezoelectric layer 502, parallel to the z-axis. In this way, the dicing kerfs 608 may separate neighboring transducer elements of transducer elements 602. For example, a dicing kerf 618 may separate a first transducer element 614 from a second transducer element 616. Similarly, a dicing kerf that is orthogonal to the dicing kerf 618 may separate the first transducer element 614 and the second transducer element 616 from neighboring transducer elements positioned at a different point along the x-axis. The transducer elements 602 may be non-limiting examples of the transducer elements 202 of FIG. 2.

The dicing kerfs 608 may extend partially through the impedance matching layer 505, relative to the z-axis. In the illustrated example, the dicing kerfs 608 may extend partially into the first sublayer 504 and may not extend into the second sublayer 506. As such, a portion of the impedance matching layer 505 may remain continuous and may provide mechanical support for transducer elements of the transducer 600. For example, a bottom surface of the first sublayer 504 of the impedance matching layer (e.g., that is in face-sharing contact with the piezoelectric layer 502 or adhesive coupling the impedance matching layer to the piezoelectric layer) may be interrupted by the dicing kerfs 608 along the y axis (and the x axis), while a top surface of the first sublayer 504 of the impedance matching layer 505 (e.g., that is in face-sharing contact with the second sublayer 506 or that faces ambient when the second sublayer is omitted) may extend continuously, without interruption by the dicing kerfs 608, along both the y axis and x axis. Further, the impedance matching layer 505 may provide ground recovery for the transducer 600. By omitting the dicing kerfs 608 from at least a portion of the impedance matching layer 505, a deposition of an additional layer or film to provide mechanical support and/or ground recovery may be omitted from a manufacturing method of the transducer 600. As such, by dicing the transducer 600 from the bottom side 620, a complexity of manufacturing and amount of manufacturing materials may be reduced.

FIG. 13 shows another cross-sectional view of the example transducer 600 after dicing. The cross-sectional view shown in FIG. 13 is taken along the cutting plane B-B′ of FIG. 3. The transducer 600 as illustrated in FIG. 13 includes the piezoelectric layer 502, the impedance matching layer 505, and the impedance dematching layer 508 of the transducer 500 of FIG. 5 as well as the dicing kerfs 608 of FIG. 6. The piezoelectric layer 502 may extend entirely across the transducer 600, relative to the x-axis.

The plurality of dicing kerfs 608 may extend into the transducer 600 from the bottom side 620 of the transducer. The dicing kerfs 608 may extend all the way through the impedance dematching layer 508 and the piezoelectric layer 502, parallel to the z-axis. In this way, the dicing kerfs 608 may separate transducer elements 602 from neighboring transducer elements. For example, a dicing kerf 1302 may separate a first transducer element 1304 from a second transducer element 1306. Similarly, a dicing kerf that is orthogonal to the dicing kerf 1302 may separate the first transducer element 1304 and the second transducer element 1306 from neighboring transducer elements positioned at a different point along the y-axis.

FIG. 7 shows a cross-sectional view of an example transducer assembly 700 including the transducer 600 and a flexible circuit 702. The flexible circuit 702 may be positioned under the transducer 600, relative to the z-axis. Further, the flexible circuit 702 may be in face sharing contact with a bottom surface of the impedance dematching layer 508 (e.g., the bottom side 620 of FIG. 6) of the transducer 600. As such, the flexible circuit 702 may be in face sharing contact with each of the transducer elements 602, such as the first transducer element 614. In this way, the transducer elements 602 may be electrically connected to element signal connections of the flexible circuit 702, such as a first element signal connection 712. The transducer elements 602 may be aligned with contact pads of the flexible circuit 702 via pick-and-place equipment in order to ensure correct alignment and reliable signal connections. Once aligned, the transducer elements 602 may be bonded to the flexible circuit 702 via epoxy (e.g., glue), anisotropic conductive particles (e.g., ACP), or anisotropic conductive film (e.g., ACF), in some examples.

The flexible circuit 702 may include ground recovery areas, such as a first ground recovery area 704 and a second ground recovery area 706. Each ground recovery area may be positioned below an end section of the transducer 600. For example, the first ground recovery area 704 is positioned below the first end section 510 and the second ground recovery area 706 is positioned below the second end section 512. As such, the ground recovery areas may electrically communicate with an electrically conductive material in the end sections of the transducer 600. The ground recovery areas may be connected to ground signal connections, such as the ground signal connection 708, which facilitate ground recovery for the transducer 600. Ground recovery of the transducer 600 may therefore occur via one or more electrically conductive materials in the impedance matching layer 505 and/or the first and second end sections, and the impedance dematching layer 508, as well as the ground recovery areas and the ground signal connections. In this way, the transducer 600 may be grounded without an additional conductive layer or film being deposited onto the transducer and manufacturing complexity may be reduced.

FIG. 16 shows a cross-sectional view of another example transducer assembly 1600 including a transducer 1601 and the flexible circuit 702 of FIG. 7. Transducer 1601 may be similar to transducer 600, other than transducer 1601 may lack the impedance dematching layer. The transducer 1601 includes a piezoelectric layer 1602. In some examples, the piezoelectric layer 1602 includes single crystal lead zirconate titante (PZT).

The transducer 1601 further includes an impedance matching layer 1605, which includes a first sublayer 1604 and the second sublayer 1606. The second sublayer 1606 may be positioned on top of the first sublayer 1604, and at least a portion of the first sublayer may be positioned on top of the piezoelectric layer 1602, relative to the z-axis. In some examples, the second sublayer 1606 may be omitted from the transducer 1601, and as such the impedance matching layer 1605 may include a single layer (e.g., the first sublayer 1604). The first sublayer 1604 and/or the second sublayer 1606 may include electrically conductive material for ground recovery of the transducer 1601.

The transducer 1601 further includes a first end section 1610 and a second end section 1612. Each of the first end section 1610 and the second end section 1612 includes ground conductive material. The piezoelectric layer 1602 may be positioned intermediate the first end section 1610 and the second end section 1612, relative to the y-axis. As such, the piezoelectric layer 1602 may not extend entirely across the transducer 1601, relative to the y-axis. Further, the piezoelectric layer 1602 may be positioned above a portion of flexible circuit 702, relative to the z-axis. A bottom surface of the piezoelectric layer 1602 may be in face sharing contact (e.g., direct contact) with a top surface of the flexible circuit 702. The first end section 1610 and the second end section 1612 may be comprised of conductive material, as explained above with respect to FIG. 5.

The transducer 1601 includes a plurality of dicing kerfs 1608. The plurality of dicing kerfs 1608 may extend into the transducer 1601 from a bottom side of the transducer. The plurality of dicing kerfs 1608 may be a non-limiting example of the dicing kerfs 208 of FIG. 2. The dicing kerfs 1608 may extend all the way through the piezoelectric layer 1602, parallel to the z-axis. In this way, the dicing kerfs 1608 may separate transducer elements from neighboring transducer elements, where each transducer element includes a portion of the piezoelectric layer 1602. For example, one of the dicing kerfs 1608 may separate a first transducer element 1614 from a second transducer element 1616. Similarly, one of the dicing kerfs 1608 may separate the first transducer element 1614 and the second transducer element 1616 from neighboring transducer elements positioned at a different point along the x-axis.

The dicing kerfs 1608 may extend partially through the impedance matching layer 1605, relative to the z-axis. In the illustrated example, the dicing kerfs 1608 may extend partially into the first sublayer 1604 and may not extend into the second sublayer 1606. As such, a portion of the impedance matching layer 1605 may remain continuous and may provide mechanical support for transducer elements of the transducer 1601. For example, a bottom surface of the first sublayer 1604 of the impedance matching layer (e.g., that is in face-sharing contact with the piezoelectric layer 1602 or adhesive coupling the impedance matching layer to the piezoelectric layer) may be interrupted by the dicing kerfs 1608 along the y axis (and the x axis), while a top surface of the first sublayer 1604 of the impedance matching layer 1605 (e.g., that is in face-sharing contact with the second sublayer 1606 or that faces ambient when the second sublayer is omitted) may extend continuously, without interruption by the dicing kerfs 1608, along both the y axis and x axis. Further, the impedance matching layer 1605 may provide ground recovery for the transducer 1601. By configuring the dicing kerfs 1608 so that the dicing kerfs terminate within the first sublayer 1604, a deposition of an additional layer or film to provide mechanical support and/or ground recovery may be omitted from a manufacturing method of the transducer 1601. As such, by dicing the transducer 1601 from the bottom side (e.g., through the piezoelectric layer 1602 first), a complexity of manufacturing and amount of manufacturing materials may be reduced.

The flexible circuit 702 may be positioned under the transducer 1601, relative to the z-axis. Further, the flexible circuit 702 may be in face sharing contact (e.g., direct contact) with a bottom surface of the piezoelectric layer 1602 of the transducer 1601. As such, the flexible circuit 702 may be in face sharing contact with each of the transducer elements, such as the first transducer element 1614. In this way, the transducer elements may be electrically connected to element signal connections of the flexible circuit 702, such as the first element signal connection 712. The transducer elements may be aligned with contact pads of the flexible circuit 702 via pick-and-place equipment in order to ensure correct alignment and reliable signal connections. Once aligned, the transducer elements may be bonded to the flexible circuit 702 via epoxy (e.g., glue), anisotropic conductive particles (e.g., ACP), or anisotropic conductive film (e.g., ACF), in some examples.

The flexible circuit 702 may include ground recovery areas, such as the first ground recovery area 704 and the second ground recovery area 706. Each ground recovery area may be positioned below an end section of the transducer 1601. For example, the first ground recovery area 704 is positioned below the first end section 1610 and the second ground recovery area 706 is positioned below the second end section 1612. As such, the ground recovery areas may electrically communicate with an electrically conductive material in the end sections of the transducer 1601. The ground recovery areas may be connected to ground signal connections, such as the ground signal connection 708, which facilitate ground recovery for the transducer 1601. Ground recovery of the transducer 1601 may therefore occur via one or more electrically conductive materials in the impedance matching layer 1605, the first end section 1610 and/or the second end section 1612, and the ground recovery areas and the ground signal connections. In this way, the transducer 1601 may be grounded without an additional conductive layer or film being deposited onto the transducer and manufacturing complexity may be reduced.

FIG. 8 shows a transducer 800 including horizontal dicing kerfs 802 and vertical dicing kerfs 804. The transducer 800 may be a non-limiting example of the transducer 600 of FIG. 6, with an impedance dematching layer, a piezoelectric layer, and an impedance matching layer stacked along the z-axis. The transducer 800 is an example of a transducer of a two-dimensional transducer probe (e.g., an ultrasound probe). As such, the transducer 800 may be used for three-dimensional (e.g., volumetric) imaging.

The horizontal dicing kerfs 802 are orthogonal to the vertical dicing kerfs 804, with the horizontal dicing kerfs being parallel to the y-axis and the vertical dicing kerfs being parallel to the x-axis. Further, the horizontal dicing kerfs 802 may extend entirely across the transducer 800, relative to the y-axis, and the vertical dicing kerfs 804 may extend entirely across the transducer, relative to the x-axis. The horizontal dicing kerfs 802 may intersect the vertical dicing kerfs 804 at 90 degree angles. The horizontal dicing kerfs 802 and the vertical dicing kerfs 804 may be non-limiting examples of the dicing kerfs 208 of FIG. 2, and as such may separate neighboring transducer elements of the transducer 800. Further, the horizontal dicing kerfs 802 and the vertical dicing kerfs 804 may only extend partially into the transducer 800, relative to the z-axis. For example, the transducer 800 may include an impedance matching layer that the dicing kerfs do not extend into.

In the illustrated example, the transducer 800 includes a first end area 806 and a second end area 808, where the second end area is positioned opposite the first end area, relative to the y-axis. Further, the transducer 800 includes a center area 810, where the center area is positioned intermediate the first end area 806 and the second end area 808. The center area 810 includes both the horizontal dicing kerfs 802 and the vertical dicing kerfs 804, while the first end area 806 and the second end area 808 may include solely the horizontal dicing kerfs 802. As such, the center area 810 may include transducer elements of the transducer 800 while the first end area 806 and the second end area 808 may not include transducer elements.

A first ground recovery area 812 is positioned in the first end area 806 and a second ground recovery area 814 is positioned in the second end area 808. The first ground recovery area 812 and the second ground recovery area 814 may facilitate ground recovery for the transducer 800. For example, ground recovery may be obtained through the electrical communication between a continuous layer of the transducer 800 (e.g., a layer that the horizontal dicing kerfs 802 and the vertical dicing kerfs 804 do not extend through) and the first ground recovery area 812 and the second ground recovery area 814. Further, the first ground recovery area 812 and the second ground recovery area 814 may be in electrical communication with a flexible circuit (e.g., the flexible circuit 702 of FIG. 7).

The one or more continuous conductive layers of the transducer 800 (e.g., the impedance matching layer) allows the first ground recovery area 812 and the second ground recovery area 814 to be positioned differently depending on the desired utilization of the transducer. For example, the positions of the first ground recovery area 812 and the second ground recovery area 814 illustrated in FIG. 8 may be beneficial for a two-dimensional transducer probe (e.g., for volumetric imaging), in some examples. In other examples, such as when the transducer 800 may be utilized in a one-dimensional probe (e.g., for two-dimensional imaging), it may be beneficial for the ground recovery areas to be positioned in a way that increases the imaging footprint parallel to a desired axis (e.g., the y-axis) may be increased.

In some examples, as illustrated in FIG. 12, a composite wafer 1200 may be diced such that dicing kerfs 1202 extend parallel to a single direction. For example, the dicing kerfs 1202, such as a first dicing kerf 1204, may be parallel to the x-axis. As such, all of the dicing kerfs 1202 may be oriented parallel to one another. In this way, the composite wafer 1200 may be separated into transducers for one-dimensional ultrasound probes configured for two-dimensional imaging.

The dicing kerfs 1202 divide a portion of the composite wafer 1200 into a plurality of transducer elements 1206, such as a first transducer element 1208 and a second transducer element 1210. Each of the transducer elements 1206 may include a portion of an impedance dematching layer 1212 stacked on top of a portion of a piezoelectric layer and a portion of an impedance matching layer, relative to the z-axis. The dicing kerfs 1202 may separate the transducer elements 1206 from neighboring transducer elements and from ground recovery areas, such as a first ground recovery area 1214. In the illustrated example, each of the transducer elements 1206 may have a rectangular cross-section parallel to the x-y plane.

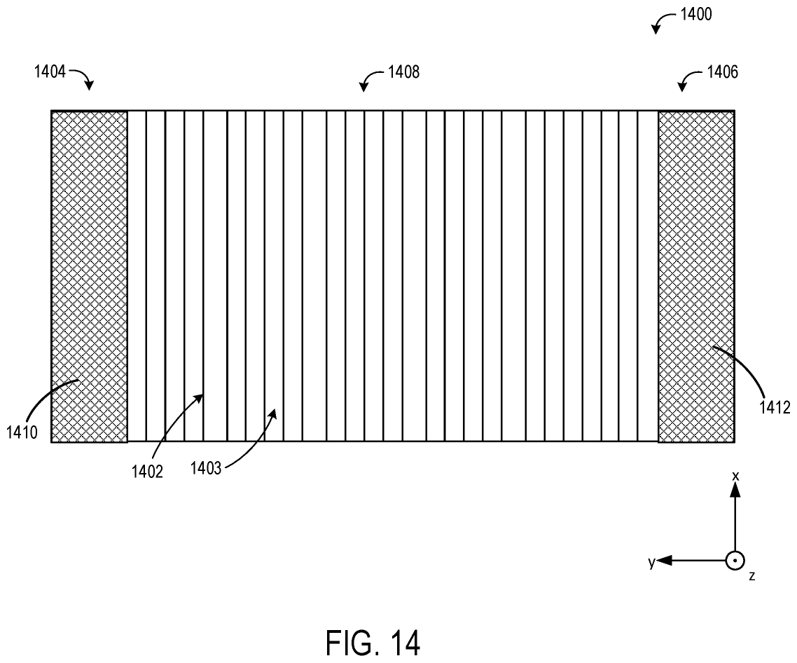

FIG. 14 shows a transducer 1400 that may be used in a one-dimensional ultrasound probe which is configured for two-dimensional imaging. The transducer 1400 may be a transducer separated from the composite wafer 1200 of FIG. 12. The transducer 1400 includes vertical dicing kerfs 1402 and may include an impedance dematching layer, a piezoelectric layer, and an impedance matching layer stacked along the z-axis.

The vertical dicing kerfs 1402 are parallel to the x-axis and extend entirely across the transducer 1400, relative to the x-axis. Further, the vertical dicing kerfs 1402 may be non-limiting examples of the dicing kerfs 208 of FIG. 2, and as such may separate neighboring transducer elements 1403 of the transducer 1400. The vertical dicing kerfs 1402 may only extend partially into the transducer 1400, relative to the z-axis, such that the vertical dicing kerfs do not extend entirely through the impedance matching layer of the transducer.

In the illustrated example, the transducer 1400 includes a first end area 1404 and a second end area 1406, where the second end area is positioned opposite the first end area, relative to the y-axis. Further, the transducer 1400 includes a center area 1408, where the center area is positioned intermediate the first end area 1404 and the second end area 1406. The center area 1408 includes the vertical dicing kerfs 1402, while the vertical dicing kerfs may be omitted from the first end area 1404 and the second end area 1406. As such, the center area 1408 may include transducer elements 1403 of the transducer 1400 while the first end area 1404 and the second end area 1406 may not include transducer elements.

A first ground recovery area 1410 is positioned in the first end area 1404 and a second ground recovery area 1412 is positioned in the second end area 1406. The first ground recovery area 1410 and the second ground recovery area 1412 may facilitate ground recovery for the transducer 1400. For example, ground recovery may be obtained through the electrical communication between a continuous layer of the transducer 1400 (e.g., a layer that the vertical dicing kerfs 804 do not extend entirely through) and the first ground recovery area 1410 and/or the second ground recovery area 1412. Further, the first ground recovery area 1410 and the second ground recovery area 1412 may be in electrical communication with a flexible circuit (e.g., the flexible circuit 702 of FIG. 7) when the transducer 1400 is included in a transducer assembly.

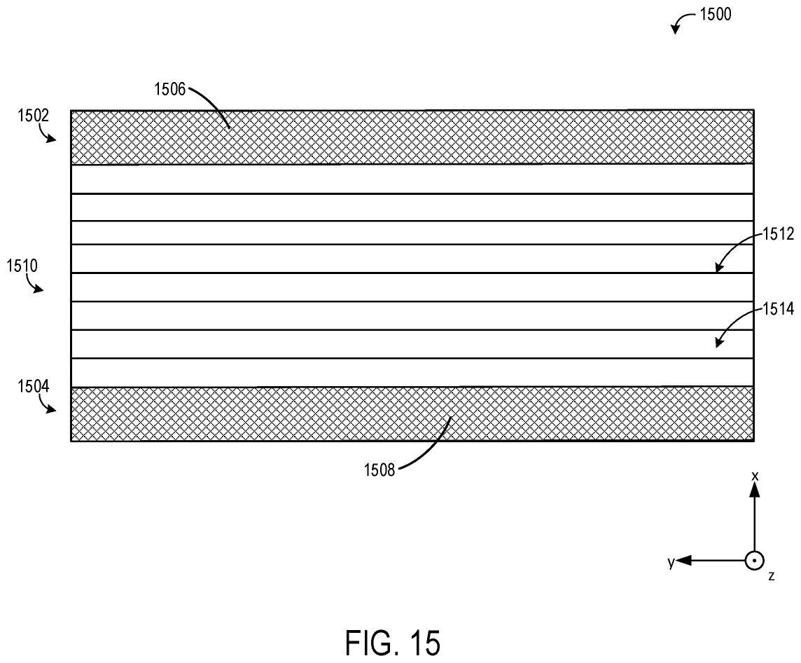

FIG. 15 illustrates a one-dimensional transducer 1500 configured with an increased imaging footprint parallel to the y-axis. The transducer 1500 may be a transducer separated from a composite wafer with dicing kerfs oriented parallel to a single direction. The transducer 1500 includes a first end area 1502 and a second end area 1504, with the second end area positioned opposite the first end area, relative to the x-axis. Further, the transducer 1500 includes a first ground recovery area 1506 positioned within the first end area 1502 and a second ground recovery area 1508 positioned within the second end area 1504. A center section 1510 of the transducer 1500 includes a plurality of dicing kerfs 1512, and each of the dicing kerfs separate transducer elements 1514 from neighboring transducer elements. The transducer elements 1514 extend entirely across the transducer 1500, relative to the y-axis, which may increase the imaging footprint of the transducer along the y-axis relative to a transducer with transducer elements that do not extend across the transducer, relative to the y-axis. For example, the transducer 1400 of FIG. 14 may have a larger imaging footprint relative to the x-axis, while the transducer 1500 of FIG. 15 may have a larger imaging footprint relative to the y-axis.

FIG. 9 illustrates a method 900 for manufacturing a one-dimensional transducer assembly (e.g., probe). The one-dimensional transducer assembly may include a transducer and a flexible circuit, and may generate two-dimensional images. The transducer may include a piezoelectric layer, an impedance matching layer, and an impedance dematching layer, stacked along a first axis. Further, edges of the transducer may extend along a second axis and a third axis, where the first axis, the second axis, and the third axis are orthogonal to one another. As such, the transducer may have a rectangular cross section parallel to a plane formed by the second axis and the third axis. The flexible circuit may be a printed circuit board (PCB), in some examples.

At 902, the method 900 includes dicing a composite wafer through an impedance dematching layer and a piezoelectric layer along a single direction. The composite wafer may include a piezoelectric layer, an impedance matching layer, and an impedance dematching layer stacked along the first axis. The piezoelectric layer may be positioned intermediate at least a portion of the impedance matching layer and at least a portion of the impedance dematching layer, relative to the first axis. Further, the piezoelectric layer may include one or more portions of piezoelectric material positioned between portions of the ground conductive material, relative to the second or third axis. Dicing of the wafer may be performed using a standard dicing saw or via laser dicing, in some examples. In other examples, a deep reactive ion or other semiconductor type etch may be used with an intervening masking step. As a result of dicing, a plurality of dicing kerfs may be introduced into the composite wafer.

The dicing kerfs may extend into the composite wafer along the first axis. Further, the dicing kerfs may extend across an entirety of the composite wafer parallel to one of the second axis or the third axis. For example, the dicing kerfs 1202 of FIG. 12 extend entirely across the composite wafer 1200, relative to the x-axis. As such, the dicing kerfs divide the wafer into a plurality of transducer elements and may separate neighboring transducer elements. The transducer elements may have a rectangular cross-section parallel to a plane formed by the second axis and the third axis. The wafer may be diced into hundreds or thousands of transducer elements, in some examples.

The composite wafer may be diced starting from the impedance dematching layer of the wafer. As such, the dicing kerfs may first be introduced to the impedance matching layer and may extend entirely through the impedance dematching layer, relative to the first axis. Further, the dicing kerfs may be introduced to the piezoelectric layer and may extend entirely through the piezoelectric layer, relative to the first axis. As such, the dicing kerfs may extend deep enough to separate neighboring transducer elements from one another, where each transducer element includes a portion of the piezoelectric layer stacked on top of a portion of the impedance dematching layer. In some examples, the dicing kerfs may then be introduced to the impedance matching layer but may extend only partially through the impedance matching layer. As such, the impedance matching layer may provide a common (e.g., continuous) mechanical support for the transducer elements. Further, the impedance matching layer may include an electrically conductive material for ground recovery of the transducer elements. As such, a conductive film and/or a conductive layer may not be deposited onto the composite wafer after dicing, reducing the number of manufacturing steps (e.g., manufacturing complexity).

At 904, the method 900 includes separating a transducer from the diced composite wafer. The composite wafer may include a plurality of transducers, with each transducer including a plurality of transducer elements. The transducer elements of each transducer may be separated from neighboring transducer elements by the dicing kerfs introduced to the composite wafer during dicing. A transducer may be removed from the composite wafer via cutting (e.g., via a saw, a laser, etc.), in some examples, and separated from the rest of the composite wafer. The separated transducer includes the piezoelectric layer, the impedance matching layer, and the impedance dematching layer, as well as a plurality of transducer elements separated by dicing kerfs.

At 906, the method 900 includes coupling the transducer to a flexible circuit. The transducer and flexible circuit are coupled together to form a transducer assembly (e.g., probe), where the flexible circuit may ground the transducer and receive signals generated by the transducer. When coupled together, the impedance dematching layer of the transducer may be in face sharing contact with one or more surfaces of the flexible circuit.

Coupling the transducer to the flexible circuit includes at 908, aligning transducer elements of the transducer with contact pads of the flexible circuit. The alignment of each transducer element with a contact pad may be facilitated by pick-and-place equipment. As such, an electrical connection (e.g., routing of signals) between the transducer and the flexible circuit may be more reliable. Coupling the transducer to the flexible circuit includes at 910, bonding the transducer elements to the contact pads. The transducer elements may be bonded to the contact pads via epoxy (e.g., glue), anisotropic conductive particles (ACP), or anisotropic conductive film (ACF), in some examples.

At 912, the method 900 optionally includes dicing the transducer through the impedance matching layer. In some examples, directivity of the transducer assembly may be increased by dicing the transducer for a second time, with a second set dicing kerfs being first introduced into the impedance matching layer of the transducer. As such, the second set of dicing kerfs may extend into the transducer along a direction parallel to the first axis and opposite to the direction that the first set of dicing kerfs extend into the transducer. The second set of dicing kerfs may extend entirely through the impedance matching layer, in some examples. As such, a conductive film or layer may be deposited onto the transducer assembly for grounding and electrical communication of transducer signals. In other examples, the second set of dicing kerfs may extend only partially through the impedance matching layer. The second set of dicing kerfs may reduce acoustic crosstalk between neighboring transducer elements.

FIG. 10 illustrates a method 1000 for manufacturing a two-dimensional transducer assembly (e.g., probe). The two-dimensional transducer assembly may include a transducer and a flexible circuit, and may generate three-dimensional images (e.g., volumetric images). The transducer may include a piezoelectric layer, an impedance matching layer, and an impedance dematching layer, stacked along a first axis. Further, edges of the transducer may extend along a second axis and a third axis, where the first axis, the second axis, and the third axis are orthogonal to one another. As such, the transducer may have a rectangular cross section parallel to a plane formed by the second axis and the third axis. The flexible circuit may be a printed circuit board (PCB), in some examples.

At 1002, the method 1000 includes dicing a composite wafer through an impedance dematching layer and a piezoelectric layer along two orthogonal directions. The composite wafer may include a piezoelectric layer, an impedance matching layer, and an impedance dematching layer stacked along the first axis. The piezoelectric layer may be positioned intermediate at least a portion of the impedance matching layer and at least a portion of the impedance dematching layer, relative to the first axis, as shown in FIG. 5. Further, the piezoelectric layer may include one or more portions of piezoelectric material positioned between portions of conductive ground material, relative to the second or third axis, as shown in FIG. 11. Dicing of the wafer may be performed using a standard dicing saw or via laser dicing, in some examples. In other examples, a deep reactive ion or other semiconductor type etch may be used with an intervening masking step. As a result of dicing, a plurality of dicing kerfs may be introduced into the composite wafer.

The dicing kerfs may extend into the composite wafer along a direction parallel to the first axis. Further, a first set of the dicing kerfs may be oriented in a first direction and a second set of the dicing kerfs may be oriented in a second direction, where the two directions are orthogonal to each other and to the first axis. For example, a first set of the dicing kerfs 208 and a second set of the dicing kerfs 208 of FIG. 2 may be orthogonal and extend entirely across the composite wafer 200, relative to the x-direction and the y-direction, respectively. In some examples, the first set of the dicing kerfs may be oriented parallel to the second axis and the second set of the dicing kerfs may be oriented parallel to the third axis. Further, the dicing kerfs may extend across an entirety of the composite wafer parallel to the second axis and the third axis. As such, the dicing kerfs divide the composite wafer into a plurality of transducer elements and may separate neighboring transducer elements. The transducer elements may have a rectangular cross-section parallel to a plane formed by the second axis and the third axis. The wafer may be diced into hundreds or thousands of transducer elements, in some examples.

The composite wafer may be diced starting from the impedance dematching layer of the wafer. As such, the dicing kerfs may first be introduced to the impedance matching layer and may extend entirely through the impedance dematching layer, relative to the first axis. Further, the dicing kerfs may be introduced to the piezoelectric layer and may extend entirely through the piezoelectric layer, relative to the first axis. As such, the dicing kerfs may extend deep enough to separate neighboring transducer elements from one another, where each transducer element includes a portion of the piezoelectric layer stacked on top of a portion of the impedance dematching layer and in contact with the impedance matching layer. In some examples, the dicing kerfs may then be introduced to the impedance matching layer but may extend only partially through the impedance matching layer. As such, the impedance matching layer may provide a common (e.g., continuous) mechanical support for the transducer elements. Further, the impedance matching layer may include an electrically conductive material for ground recovery of the transducer elements. As such, a conductive film and/or a conductive layer may not be deposited onto the composite wafer after dicing, reducing the number of manufacturing steps (e.g., manufacturing complexity).

At 1004, the method 1000 includes separating a transducer from the diced composite wafer. At 1006, the method 1000 includes coupling the transducer to a flexible circuit. Coupling the transducer to the flexible circuit includes at 1008, aligning transducer elements of the transducer with contact pads of the flexible circuit. Further, coupling the transducer to the flexible circuit includes at 1010, bonding the transducer elements to the contact pads. At 1012, the method 1000 optionally includes dicing the transducer through an impedance matching layer. 1004, 1006, 1008, 1010, and 1012 may be identical to 904, 906, 908, 910, and 912 of FIG. 9, respectively. It is to be appreciated that for transducers that lack an impedance dematching layer (e.g., transducer 1601 of FIG. 16), methods 900 and 1000 may be performed as described above, but with the dicing of the composite wafer starting at the piezoelectric layer and with the resultant transducer coupled to the flexible circuit such that the piezoelectric layer is coupled to the flexible circuit rather than the impedance dematching layer.

The disclosure also provides support for a transducer assembly, comprising: a flexible circuit, and a transducer including a piezoelectric layer and an impedance matching layer, wherein the transducer comprises a plurality of transducer elements formed via a plurality of dicing kerfs positioned between neighboring transducer elements of the plurality of transducer elements, and each dicing kerf of the plurality of dicing kerfs extends only partially through the impedance matching layer. In a first example of the assembly, the assembly further comprises: an impedance dematching layer, wherein the impedance dematching layer is in face sharing contact with one or more surfaces of the flexible circuit. In a second example of the assembly, optionally including the first example, each dicing kerf of the plurality of dicing kerfs extends entirely through the piezoelectric layer and at least a portion of the impedance dematching layer. In a third example of the assembly, optionally including one or both of the first and second examples, the plurality of transducer elements is bonded to contact pads of the flexible circuit. In a fourth example of the assembly, optionally including one or more or each of the first through third examples, the plurality of transducer elements is bonded to the contact pads via epoxy. In a fifth example of the assembly, optionally including one or more or each of the first through fourth examples, the plurality of dicing kerfs includes a first set of dicing kerfs parallel to a first axis and a second set of dicing kerfs parallel to a second axis, where the second axis is orthogonal to the first axis. In a sixth example of the assembly, optionally including one or more or each of the first through fifth example, the piezoelectric layer is in face sharing contact with one or more surfaces of the flexible circuit and wherein each dicing kerf of the plurality of dicing kerfs extends entirely through the piezoelectric layer. In a seventh example of the assembly, optionally including one or more or each of the first through sixth examples, at least a portion of the impedance matching layer is continuous from a first end of the transducer to a second end of the transducer. In an eighth example of the assembly, optionally including one or more or each of the first through seventh examples, the impedance matching layer includes an electrically conductive material. In a ninth example of the assembly, optionally including one or more or each of the first through eighth examples, the impedance matching layer includes a first sublayer and a second sublayer, wherein at least a portion of the first sublayer is positioned intermediate the second sublayer and the piezoelectric layer. In a tenth example of the assembly, optionally including one or more or each of the first through ninth examples, the system further comprises: a first ground recovery area at a first side of the transducer and a second ground recovery area at a second side of the transducer. In an eleventh example of the assembly, optionally including one or more or each of the first through tenth examples, each of the first ground recovery area and second ground recovery area is aligned with respective ground signal connections of the flexible circuit.

The disclosure also provides support for a method for manufacturing a transducer assembly, comprising: dicing a transducer to form dicing kerfs that separate transducer elements from neighboring transducer elements, aligning the transducer elements with contact pads of a flexible circuit, and bonding the transducer to the flexible circuit. In a first example of the method, dicing the transducer comprises dicing the transducer through an impedance dematching layer and a piezoelectric layer of the transducer, such that the dicing kerfs extend entirely through the impedance dematching layer and the piezoelectric layer of the transducer, relative to a first axis. In a second example of the method, optionally including the first example, dicing the transducer comprises dicing the transducer partially through an impedance matching layer of the transducer such that the dicing kerfs extend only partially into the impedance matching layer, relative to a first axis. In a third example of the method, optionally including one or both of the first and second examples, aligning the transducer elements with contact pads of the flexible circuit comprises aligning the transducer elements with the contact pads via pick-and-place equipment. In a fourth example of the method, optionally including one or more or each of the first through third examples, the method further comprises: dicing one or more additional transducers collectively with the transducer and separating the transducer from the one or more additional transducers. In a fifth example of the method, optionally including one or more or each of the first through fourth examples, dicing the transducer comprises dicing the transducer such that a first set of the dicing kerfs are oriented parallel to a second axis and dicing the transducer such that a second set of the dicing kerfs are oriented parallel to a third axis, wherein the second axis and the third axis are orthogonal to one another.

The disclosure also provides support for a transducer system, comprising: a transducer including a piezoelectric layer, an impedance matching layer, a first conductive end section, and a second conductive end section, wherein the piezoelectric layer is positioned intermediate the first conductive end section and the second conductive end section, and a flexible circuit, wherein a bottom surface of the transducer is directly coupled to contact pads of the flexible circuit. In a first example of the system, the transducer includes dicing kerfs that extend entirely through the piezoelectric layer and partially through the impedance matching layer, wherein at least a portion of the impedance matching layer is continuous, and wherein the bottom surface comprises a bottom surface of the piezoelectric layer or a bottom surface of an impedance dematching layer.

The technical effect of a transducer assembly manufacturing process that includes dicing entirely through the impedance dematching layer and the piezoelectric, and not entirely through the impedance matching layer is that dicing may be performed collectively at a wafer level rather than individually at a circuit level. Further, at least a portion of the impedance matching layer remains continuous, providing mechanical stability and ground recovery without an additional conductive layer being bonded to the transducer assembly.

FIGS. 1-8 and 11-16 show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below/underneath one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A transducer assembly, comprising:
a flexible circuit; and
a transducer including a piezoelectric layer and an impedance matching layer;
wherein the transducer comprises a plurality of transducer elements formed via a plurality of dicing kerfs positioned between neighboring transducer elements of the plurality of transducer elements; and
wherein each dicing kerf of the plurality of dicing kerfs extends only partially through the impedance matching layer.

2. The transducer assembly of claim 1, further comprising an impedance dematching layer, wherein the impedance dematching layer is in face sharing contact with one or more surfaces of the flexible circuit.

3. The transducer assembly of claim 2, wherein each dicing kerf of the plurality of dicing kerfs extends entirely through the piezoelectric layer and at least a portion of the impedance dematching layer.

4. The transducer assembly of claim 1, wherein the plurality of transducer elements is bonded to contact pads of the flexible circuit.

5. The transducer assembly of claim 4, wherein the plurality of transducer elements is bonded to the contact pads via epoxy.

6. The transducer assembly of claim 1, wherein the plurality of dicing kerfs includes a first set of dicing kerfs parallel to a first axis and a second set of dicing kerfs parallel to a second axis, where the second axis is orthogonal to the first axis.

7. The transducer assembly of claim 1, wherein the piezoelectric layer is in face sharing contact with one or more surfaces of the flexible circuit and wherein each dicing kerf of the plurality of dicing kerfs extends entirely through the piezoelectric layer.

8. The transducer assembly of claim 1, wherein at least a portion of the impedance matching layer is continuous from a first end of the transducer to a second end of the transducer.

9. The transducer assembly of claim 8, wherein the impedance matching layer includes an electrically conductive material.

10. The transducer assembly of claim 1, wherein the impedance matching layer includes a first sublayer and a second sublayer, wherein at least a portion of the first sublayer is positioned intermediate the second sublayer and the piezoelectric layer.

11. The transducer assembly of claim 1, further comprising a first ground recovery area at a first side of the transducer and a second ground recovery area at a second side of the transducer.

12. The transducer assembly of claim 11, wherein each of the first ground recovery area and second ground recovery area is aligned with respective ground signal connections of the flexible circuit.

13. A method for manufacturing a transducer assembly, comprising:
dicing a transducer to form dicing kerfs that separate transducer elements from neighboring transducer elements;
aligning the transducer elements with contact pads of a flexible circuit; and
bonding the transducer to the flexible circuit.

14. The method of claim 13, wherein dicing the transducer comprises dicing the transducer through an impedance dematching layer and a piezoelectric layer of the transducer, such that the dicing kerfs extend entirely through the impedance dematching layer and the piezoelectric layer of the transducer, relative to a first axis.

15. The method of claim 13, wherein dicing the transducer comprises dicing the transducer partially through an impedance matching layer of the transducer such that the dicing kerfs extend only partially into the impedance matching layer, relative to a first axis.

16. The method of claim 13, wherein aligning the transducer elements with contact pads of the flexible circuit comprises aligning the transducer elements with the contact pads via pick-and-place equipment.

17. The method of claim 13, further comprising dicing one or more additional transducers collectively with the transducer and separating the transducer from the one or more additional transducers.

18. The method of claim 13, wherein dicing the transducer comprises dicing the transducer such that a first set of the dicing kerfs are oriented parallel to a second axis and dicing the transducer such that a second set of the dicing kerfs are oriented parallel to a third axis, wherein the second axis and the third axis are orthogonal to one another.

19. A transducer system, comprising:
a transducer including a piezoelectric layer, an impedance matching layer, a first conductive end section, and a second conductive end section, wherein the piezoelectric layer is positioned intermediate the first conductive end section and the second conductive end section; and
a flexible circuit, wherein a bottom surface of the transducer is directly coupled to contact pads of the flexible circuit.

20. The transducer system of claim 19, wherein the transducer includes dicing kerfs that extend entirely through the piezoelectric layer and partially through the impedance matching layer, wherein at least a portion of the impedance matching layer is continuous, and wherein the bottom surface comprises a bottom surface of the piezoelectric layer or a bottom surface of an impedance dematching layer.
